(19) United States
(12) Patent Application Publication
(10) Pub. No.: US 2026/0294515 A1
(43) Pub. Date: Oct. 1, 2026

(54) ENERGY-BASED SURGICAL INSTRUMENTS INCLUDING LOCAL GENERATOR

(71) Applicant: Cilag GmbH International, Zug (CH)

(72) Inventors: Frederick E. Shelton, IV, Hillsboro, OH (US); Jacqueline C. Aronhalt, Loveland, OH (US); Jason L. Harris, Lebanon, OH (US); Shane R. Adams, Lebanon, OH (US)

(21) Appl. No.: 19/366,978

(22) Filed: Oct. 23, 2025

Related U.S. Application Data
(60) Provisional application No. 63/780,371, filed on Mar. 30, 2025.

(51) Int. Cl. A61B 18/14; A61B 18/00; A61B 18/12

(52) U.S. Cl. CPC A61B 18/1445; A61B 2018/00208; A61B 2018/00601; A61B 2018/00607; A61B 2018/0063; A61B 2018/00642; A61B 2018/00702; A61B 2018/00779; A61B 2018/1226; A61B 2018/1273

(57) ABSTRACT

A surgical system includes an energy-based surgical instrument including a local generator and a remote generator separate from the energy-based surgical instrument. The energy-based surgical instrument has an energy mode that, in response to activation, delivers an amount of energy to a patent tissue. The remote generator is configured to generate a first amount of energy for the energy mode of the energy-based surgical instrument, and the local generator is configured to generate a second amount of energy, less than the first amount of energy of the remote generator, for the energy model. The energy-based surgical instrument may include a controller configured to determine whether to use the remote generator or the local generator for activation of the energy mode of the energy-based surgical instrument. The energy-based surgical instrument may also include a power accumulator, which may be charged using energy provided by the remote generator.

This application claims the benefit of and priority to U.S. Patent Application No. 63/780,371, entitled “ENERGY-BASED SURGICAL INSTRUMENT INCLUDING LOCAL GENERATOR,” which was filed on Mar. 30, 2025, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient's tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient's tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a surgical system for performing an energy-based surgical procedure includes an energy-based surgical instrument and a remote generator. The energy-based surgical instrument may have an energy mode that, in response to activation, delivers an amount of energy to a patient tissue. The remote generator may be separate from the energy-based surgical instrument and may be configured to generate a first amount of energy for the energy mode of the energy-based surgical instrument. The energy-based surgical instrument may include a local generator and a controller. The local generator may be configured to generate a second amount of energy, less than the first amount of energy of the remote generator, for the energy mode. The controller may be configured to monitor for a request to activate the energy mode of the energy-base surgical instrument, determine whether to use the remote generator or the local generator to activate the energy mode, and activate the energy mode of the energy-based surgical instrument using the determined generator.

In some embodiments, to determine whether to use the remote generator or the local generator to activate the energy mode may include to determine whether to use the remote generator or the local generator to activate the energy mode based on the energy-based surgical procedure being performed. Additionally or alternatively, to determine whether to use the remote generator or the local generator to activate the energy mode may include to determine whether to use the remote generator or the local generator to activate the energy mode based a sensed characteristic of the patient tissue.

Additionally, in some embodiments, to determine whether to use the remote generator or the local generator to activate the energy mode may include to determine to use the remote generator to activate the energy mode. In such embodiments, the controller may be further configured to monitor the operation of the remote generator; determine, based on the operation of the remote generator, whether to supplement the first amount of energy generated by the remote generator using the second amount of energy generated by the local generator; and supplement, in response to a determination to supplement the first amount of energy generated by the remote generator, the first amount of energy generated by the remote generator with the second amount of energy generated by the local generator during the activation of the requested energy mode.

Additionally or alternatively, to determine whether to use the remote generator or the local generator to activate the energy mode may include to determine to use the remote generator to activate the energy mode. In such embodiments, the controller may be further configured to monitor the operation of the remote generator; determine whether to perform a supplemental function of the energy-based surgical instrument using the second amount of energy generated by the local generator; and perform the supplemental function using the second amount of energy generated by the local generator in response to a determination to perform the supplemental function. Additionally, in such embodiments, to determine whether to perform the supplemental function of the energy-based surgical instrument may include to determine whether to perform the supplemental function of the energy-based instrument based on a sensed characteristic of the patient tissue. Additionally or alternatively, to determine whether to perform the supplemental function of the energy-based surgical instrument may include to determine whether to perform the supplemental function of the energy-based instrument based on the operation of the remote generator.

In some embodiments, to determine whether to use the remote generator or the local generator to activate the energy mode may include to determine to use the local generator to activate the energy mode. In such embodiments, the controller may be further configured to monitor the operation of the local generator; determine, based on the operation of the local generator, whether to supplement the second amount of energy generated by the local generator using the first amount of energy generated by the remote generator; and supplement, in response to a determination to supplement the second amount of energy generated by the local generator, the second amount of energy generated by the local generator with the first amount of energy generated by the remote generator during the activation of the requested energy mode.

Additionally or alternatively, in some embodiments, to determine whether to use the remote generator or the local generator to activate the energy mode may include to determine to use the local generator to activate the energy mode. In such embodiments, the controller may be further configured to monitor the operation of the local generator; determine whether to perform a supplemental function of the energy-based surgical instrument using the first amount of energy generated by the remote generator; and perform the supplemental function using the first amount of energy generated by the remote generator in response to a determination to perform the supplemental function. In such embodiments, to determine whether to perform the supplemental function of the energy-based surgical instrument may include to determine whether to perform the supplemental function of the energy-based instrument based on a sensed characteristic of the patient tissue. Additionally or alternatively, in such embodiments, to determine whether to perform the supplemental function of the energy-based surgical instrument may include to determine whether to perform the supplemental function of the energy-based instrument based on the operation of the local generator. Further, in such embodiments, the second amount of energy generated by the local generator may be used to seal the patient tissue and the first amount of energy generated by the remote generator is used to cut the patient tissue.

In some embodiments, the energy-based surgical instrument may further include a power accumulator configured to store an amount of power. In such embodiments, the controller may be configured to charge the power accumulator using a subset of the first energy provided by the remote generator. Additionally, in such embodiments, the power accumulator may be embodied as a super capacitor. For example, the super capacitor may have a capacitance between 0.5 Farads and 2 Farads.

Additionally, in such embodiments, the controller may be configured to determine whether to charge the power accumulator based on an amount of available energy provided by the remote generator. Additionally or alternatively, the controller may be further configured to monitor power requirements of the energy-based surgical instrument and perform a function of the energy-based surgical instrument using power provided by the power accumulator based on the power requirements of the energy-based surgical instrument. In such embodiments, to perform the function of the energy-based surgical instrument may include to operate jaw motors of a jaw assembly of the energy-based surgical instrument using the power provided by the power accumulator.

In some embodiments, the energy-based surgical instrument may further include a local power source. In such embodiments, to perform the function of the energy-based surgical instrument may include to supplement the power provided by the power accumulator with power provided by the local power source. Additionally, in such embodiments, to perform the function of the energy-based surgical instrument may include to operate jaw motors of a jaw assembly of the energy-based surgical instrument using the power provided by the power accumulator and the power provided by the local power source.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a block diagram of another embodiment of a control circuit of the surgical instrument of FIG. 2 including a local generator and a power accumulator;

FIGS. 9 and 10 is a simplified flow diagram of a method for controlling an energy-based surgical instrument using energy generated by a remote generator and/or a local generator, which may be executed by the controller of FIG. 8;

FIG. 11 is a simplified flow diagram of a method for charging the power accumulator of the control circuit of FIG. 8; and

FIG. 12 is a simplified flow diagram of a method for performing a function of the surgical instrument of FIGS. 2 and 8 using power from the power accumulator of the control circuit of FIG. 8.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

terms representing anatomical references, such as anterior, posterior, medial, lateral, superior, inferior, distal, proximal, et cetera, may be used throughout the specification in reference to the surgical instruments described herein as well as in reference to the patient's natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

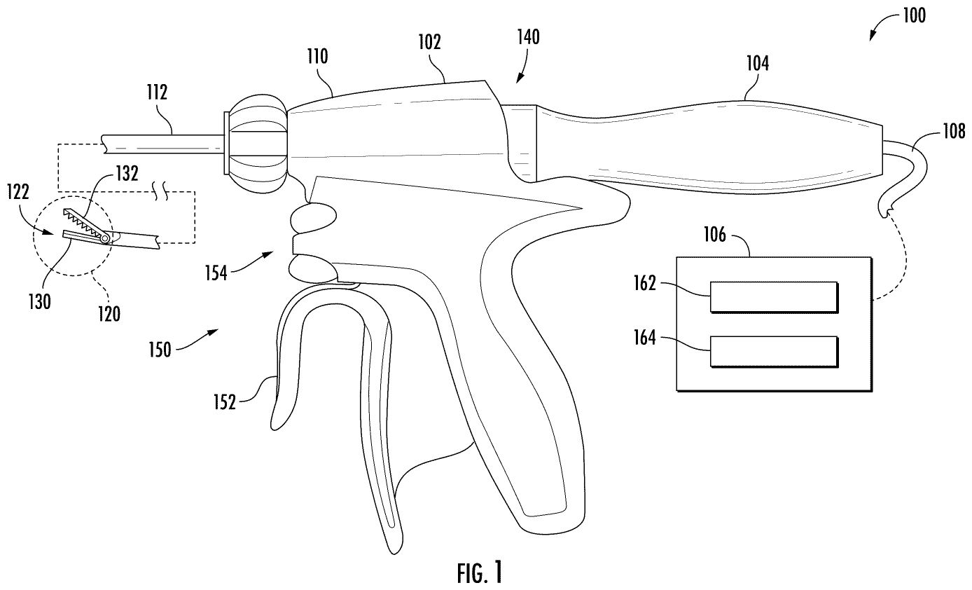

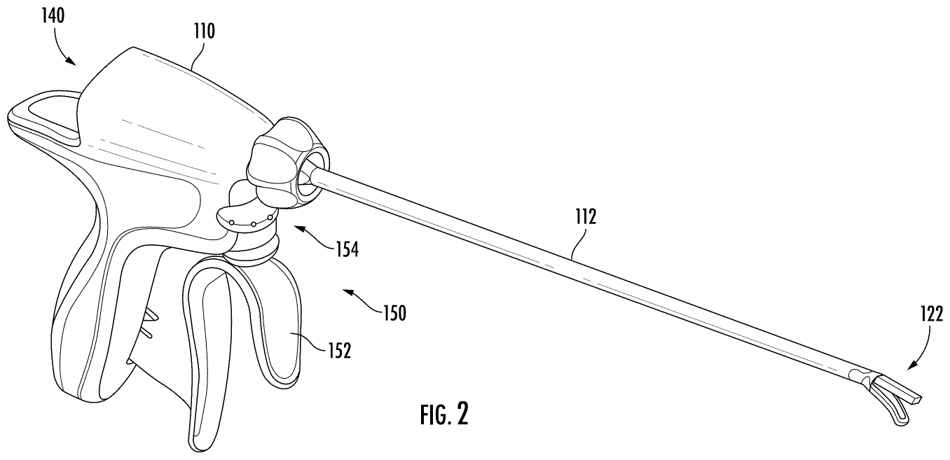

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

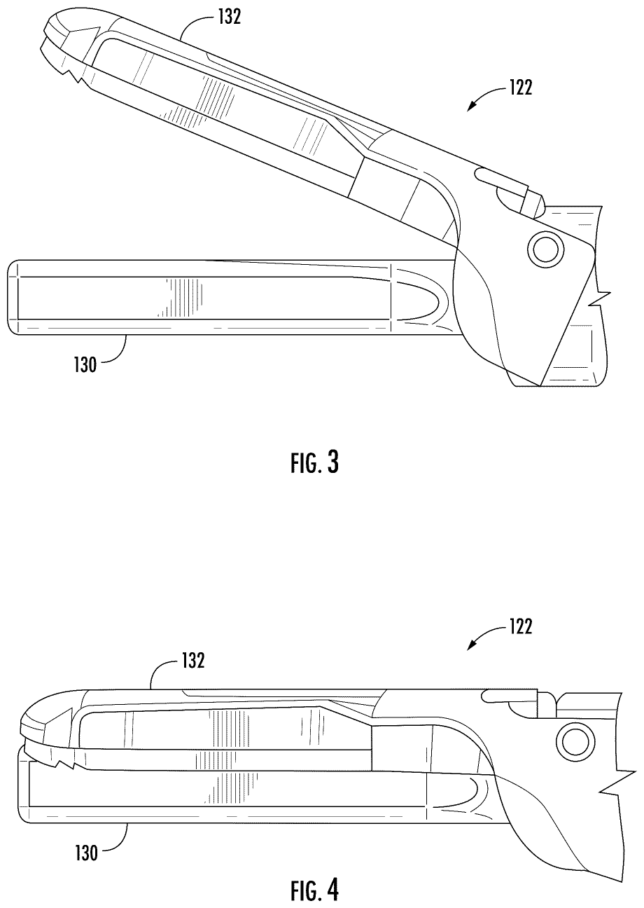

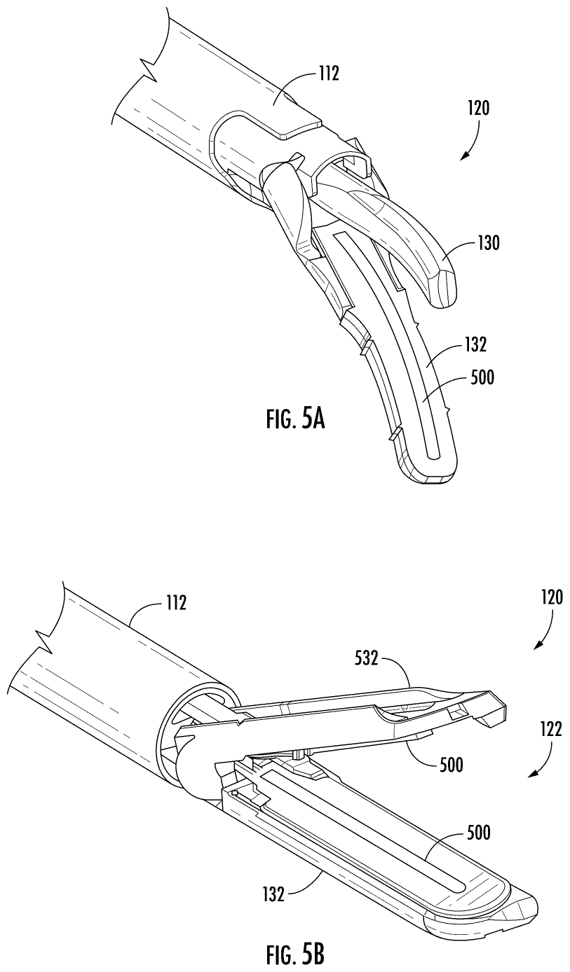

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient's body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient's tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient's tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient's tissue. For example, the electro-surgical generator module 164 may be configured to measure an impedance of the patient's tissue using the electrodes 500 and a suitable RF energy level.

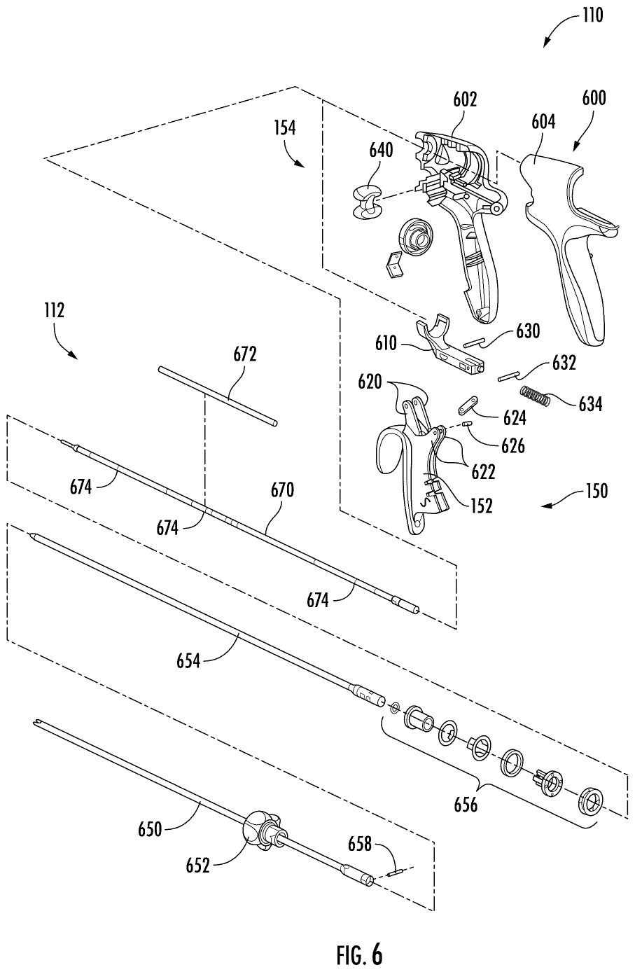

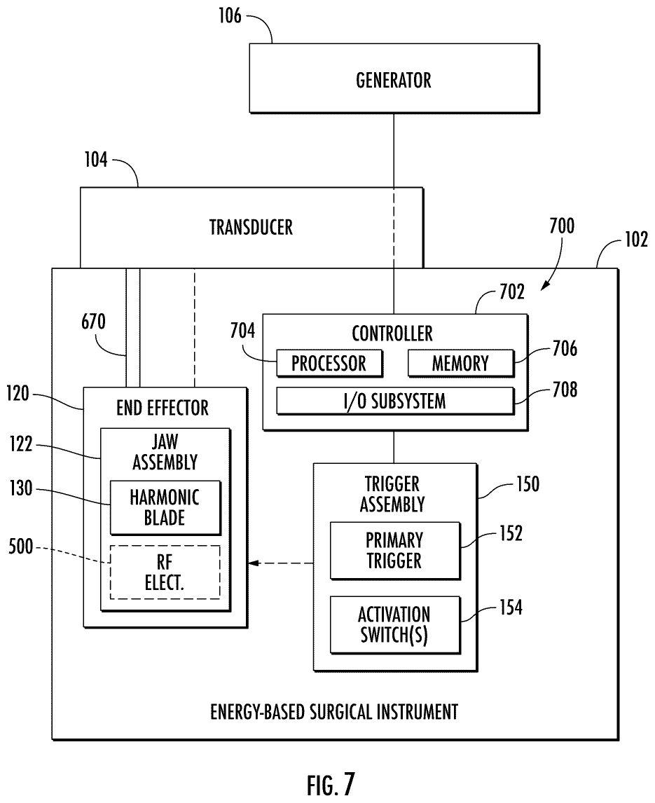

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

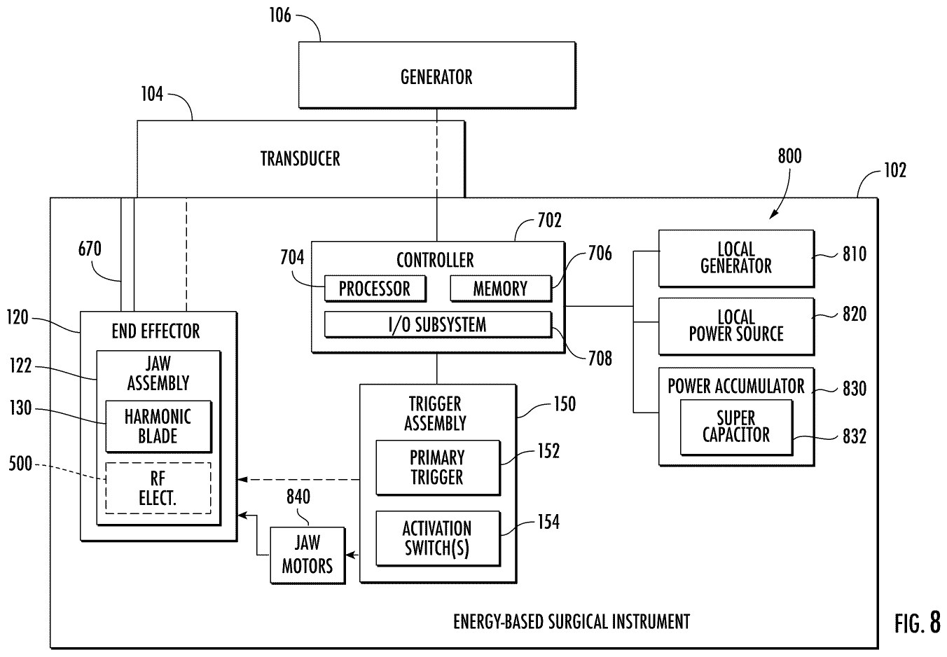

Referring now to FIG. 8, in another embodiment, the surgical instrument 102 includes a local generator 810 and is configured to determine whether to utilize energy generated by the remote generator 106 and/or by the local generator 810 to perform a requested energy mode. For example, in some embodiments as described below, the surgical instrument 102 may be configured to supplement energy provided by either the remote generator 106 or the local generator 810 with energy provided by the other one of the remote generator 106 or the local generator 810. Typically, the energy generated and provided by the local generator 810 is less than the energy generated by the remote generator 106. In addition to supplementing the provided energy, the surgical instrument 102 may utilize the energy generated by the generator 106, 810 that is not presently performing a requested energy mode to perform other supplemental functions of the surgical instrument 102. For example, in some embodiments, the local generator 810 may be used to generate RF energy to seal patient tissue, while the remote generator 106 is used to generate harmonic energy to cut the patient tissue using the harmonic blade 130. As such, various combinations of the energy provided by each of the generators 106, 810 may be used to perform the same or associated functions.

In such embodiments, the surgical instrument 102 may include a control circuit 800 as shown in FIG. 8. The control circuit 800 is substantially similar to the control circuit 700 described above and components of the control circuit 800 that correspond to similar components of control circuit 700 have been identified using the same reference number in FIG. 8 with the understanding that the description of such components provided above with regard to FIG. 7 applies to the corresponding components of the control circuit 800.

As shown in FIG. 8, the control circuit 800 includes the local generator 810, a local power source 820, and a power accumulator 830. Similar to the remote generator 106, the local generator 810 is configured to generate an amount of energy for an energy mode of the energy-based surgical instrument 102. The remote generator 106 may be configured to operate in different modes including an ultrasonic mode for providing energy to the harmonic blade 130 and/or an electro-surgical/Radio Frequency (RF) mode for providing energy to the RF electrodes 500 similar to the functionality of the remote generator 106 described above. The various modes of the local generator 810 may be operated independently of each other in some embodiments. For example, the local generator 810 may activate an ultrasonic mode to apply ultrasonic energy to the harmonic blade 130 and subsequently, either therapeutic or sub-therapeutic RF energy may be applied to the RF electrode(s) 500. Alternatively, the activation modes of the local generator 810 may be operated simultaneously or contemporaneously with each other. As described in more detail below, the energy produced by the local generator 810 may be used to supplement the energy produced by the remote generator 106 and vice-versa. Additionally or alternatively, the energy produced by the local generator 810 may be used to perform supplemental or other functions different form the energy mode and/or function performed using the energy generated by the remote generator 106.

The local power source 820 may be embodied as any type of electrical power source capable of providing power to the other components of the energy-based surgical instrument 102. For example, in the illustrative embodiment, the local power source 820 may be embodied as a battery, a set of batteries, or similar independent power source. In some embodiments, the local power source 820 may be rechargeable (e.g., rechargeable battery) and may be recharged using power provided by the remote generator 106. It should be appreciated that the local power source 820 may provide power to the local generator 810 in some embodiments. In other embodiments, the local generator 810 is powered by power provided by the remote generator 106. In such embodiments, the local power source 820 may or may not provide supplemental power to the local generator 810.

The power accumulator 830 may be embodied as any type of component, device, or collection thereof capable of accumulating and storing power over a time period. For example, in the illustrative embodiment, the power accumulator 830 is embodied as or otherwise includes a super capacitor 832. The super capacitor 832 may, for example, have a capacitance in the range of 0.5 Farads to 2.0 Farads. In use, as described in more detail below, the controller 702 is configured to charge the power accumulator 830 using available (e.g., surplus) power provided by the remote generator 106. When sufficiently charged, the power accumulator 830 may provide supplemental power to various components of the energy-based surgical instrument 102, which may allow the energy-based surgical instrument 102 to be used periodically without connection to the remote generator 106. For example, in some embodiments, the power provided by the power accumulator 830 may be used to drive components of the energy-based surgical instrument 102 for which the local power source 820 is unable to provide sufficient power (e.g., the jaw motors 840 used to control the jaw assembly 122). In such embodiments, the controller 702 may control the power accumulator 830 to provide the required power, which may supplement power from the local power source 820 and/or power provided by the remote generator 106. For example, the controller 702 may be configured to utilize the power provided by the power accumulator 830 while power provided by the remote generator 106 is utilized for other functionality of the energy-based surgical instrument 102.

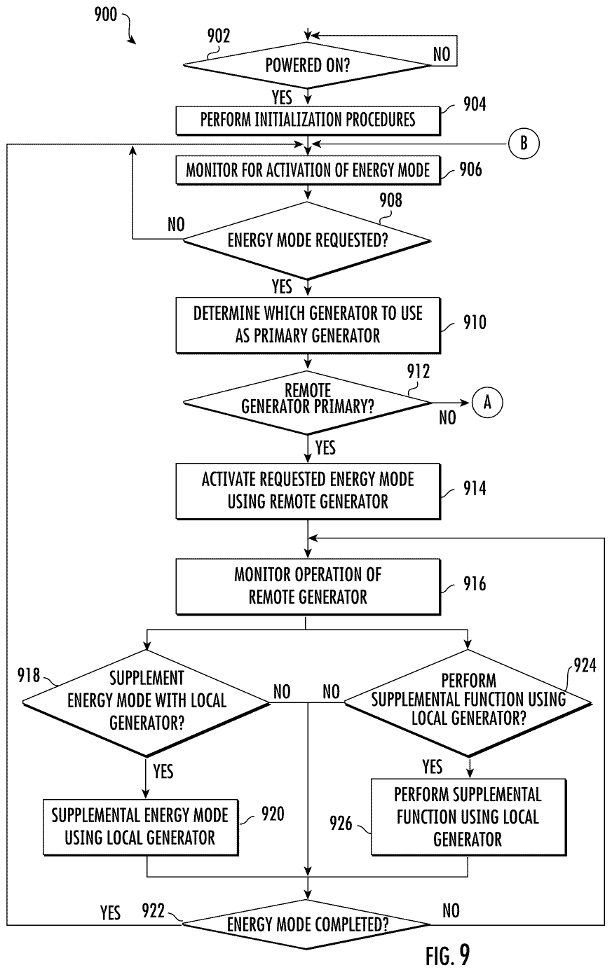

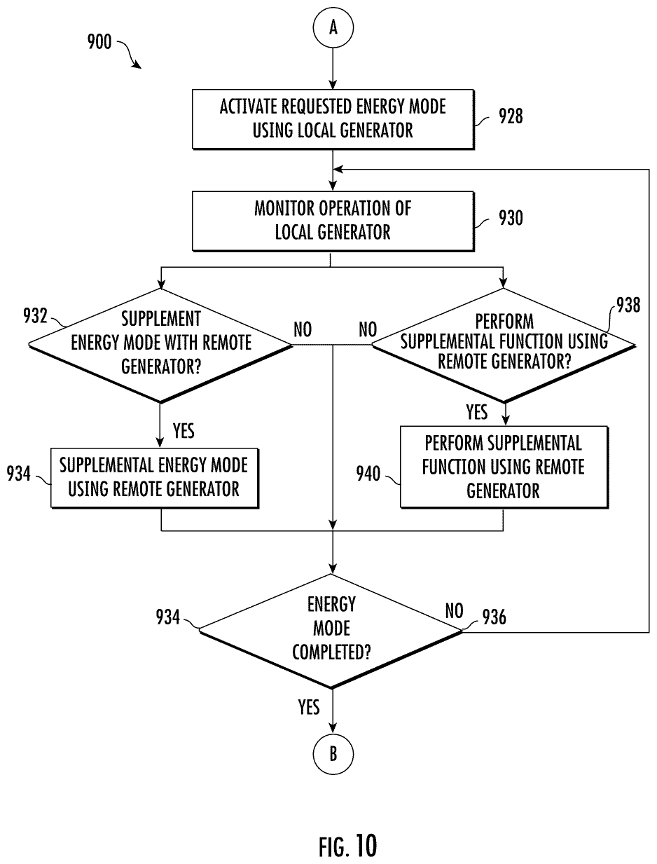

Referring now to FIGS. 9 and 10, in use, the controller 702 of the energy-based surgical instrument 102 may execute a method 900 for controlling operation of the energy-based surgical instrument 102 using energy generated by the remote generator 106 and/or the local generator 810. The method begins with block 902 in which controller 702 determines whether the energy-based surgical instrument 102 has been powered to an “on” state. If so, the method 900 advances to block 904 in which the controller 702 performs one or more initialization procedures. For example, the controller 702 may verify operation of the local generator 810, connection of the transducer 104 and/or remote generator 106, and/or other initialization or verification procedure.

After the controller 702 has performed the initialization procedures in block 904, the method 900 advances to block 906 in which the controller 702 monitors for a request for the activation of an energy mode of the energy-based surgical instrument 102. For example, the surgeon may request activation of an ultrasonic mode and/or an RF mode by operating a corresponding switch of the switch assembly 154 of the trigger assembly 150, as discussed above. In some embodiments, the switch assembly 154 may include a dedicated switch for controlling activation of each the remote generator 106 and the local generator 810 (and the different modes of each generator 106, 810).

If the controller 702 detects a request for an energy mode of the energy-based surgical instrument 102 in block 908, the method 900 advances to block 910. In block 910, the controller 702 determines whether to use the remote generator 106 or the local generator 810 for the requested energy mode. To do so, the controller 702 may consider any suitable criteria. For example, in some embodiments, the controller 702 may be configured to determine whether to use the remote generator 106 or the local generator 810 for the requested energy mode based on the energy-based surgical procedure being performed (e.g., based on the amount of energy and/or time required for one or more sub-procedures of the energy-based surgical procedure). Additionally or alternatively, the controller 702 may be configured to determine whether to use the remote generator 106 or the local generator 810 for the requested energy mode based one or more sensed characteristics of the patient tissue (e.g., a tissue impedance, temperature, compression amount, etc.).

Subsequently, in block 912, the controller 702 determines whether the remote generator 106 was selected in block 910 as the “primary” generator for the requested energy mode. If so, the method 900 advances to block 914 in which the controller 702 activates the requested energy mode using energy provided by the remote generator 106. For example, the controller 702 may determine that an ultrasonic energy mode has been requested and that the remote generator 106 is the “optimal” or better generator to provide the ultrasonic energy mode in the present circumstances. In such embodiments, the controller 702 activates the requested ultrasonic energy mode using the energy provided by the remote generator 106.

After the controller 702 has activated the requested energy mode using the energy provided by the remote generator 106 in block 914, the method 900 advances to block 916. In block 916, the controller 702 monitors operation of the remote generator 106. That is, the controller 702 monitors the performance of the requested energy mode provided by the remote generator 106. Such monitoring may include, for example, monitoring the amount or level or energy provided by the remote generator 106, monitoring the process steps performed by the remote generator 106 during the requested energy mode, and/or other operational characteristics of the remote generator 106 and/or the energy provided by the remote generator 106.

While monitoring the operation of the remote generator 106 in block 916, the controller 702 determines whether to supplement the energy mode performed by the remote generator 106 with energy provided by the local generator 810 in block 918. For example, the controller 702 may monitor the sufficiency of the energy provided by the remote generator 106 throughout the performance of the requested energy mode and determine to supplement the provided energy should the provided energy be below a reference threshold. If so, the method 900 advances to block 920 in which the controller 702 supplements the energy mode provided by the remote generator 106 with energy provided by the local generator 810. For example, the local generator 810 may operate in a series fashion with the remote generator 106 to provide additional energy for the requested energy mode.

After the controller 702 has supplemented the energy mode with energy from the local generator 810, the method 900 advances to block 922 in which the controller 702 determines whether the energy mode is completed. If not, the method 900 loops back to block 916 in which the controller 702 continues to monitor the operation of the remote generator 106. If, however, the requested energy mode is completed, the method 900 loops back to block 906 in which the controller 702 continues to monitor for a request for activation of an energy mode of the energy-based surgical instrument 102.

Referring back to block 916, while monitoring the operation of the remote generator 106, the controller 702 also determines whether to perform a supplemental function of the energy-based surgical instrument 102 using energy provided by the local generator 810 in block 924. For example, the controller 702 may be configured to determine whether to perform the supplemental function of the energy-based surgical instrument 102 based on a sensed characteristic of the patient tissue (e.g., a tissue impedance, temperature, and/or compression). Additionally or alternatively, the controller 702 may be configured to determine whether to perform the supplemental function of the energy-based surgical instrument 102 based on an operation characteristic of the remote generator 106 (e.g., the usage level of the output of the remote generator 106).

If the controller 702 determines to perform the supplemental function using the energy provided by the local generator 810 in block 924, the method 900 advances to block 926. In block 926, the controller 702 performs the supplemental function using the energy provided by the local generator 810. For example, the controller 702 may be configured to utilize the energy provided by the local generator 810 to seal the patient's tissue while the energy provided by the remote generator 106 is used to cut the patient's tissue. In such embodiments, the end effector 120 of the energy-based surgical instrument 102 may include both the harmonic blade 130 and the RF electrode(s) 500, and controller 702 may control operation of the harmonic blade 130 using the energy provided by the remote generator 106 to transect the patient's tissue and control operation of the RF electrode(s) 500 using the energy provided by the local generator 810 to contemporaneously seal the patient's tissue. Additionally or alternatively, the controller 702 may utilize the energy provided by the local generator 810 to perform various sub-therapeutic functions of the energy-based surgical instrument 120 such as tissue impedance determination.

After the controller 702 has performed the supplemental function using the energy provided by the local generator 810 in block 926, the method 900 advances to block 922. As discussed above, the controller 702 determines whether the energy mode is completed. If not, the method 900 loops back to block 916 in which the controller 702 continues to monitor the operation of the remote generator 106. If, however, the requested energy mode is completed, the method 900 loops back to block 906 in which the controller 702 continues to monitor for a request for activation of an energy mode of the energy-based surgical instrument 102.

Referring now back to block 912, if the controller 702 determines the remote generator 106 was not selected as the “primary” generator for the requested energy mode (i.e., the local generator 810 was selected), the method 900 advances to block 928 of FIG. 10. In block 928, the controller 702 activates the requested energy mode using energy provided by the local generator 810. For example, the controller 702 may determine that electro-surgical/RF mode has been requested and that the local generator 810 is the “optimal” or better generator to provide the electro-surgical/RF energy mode in the present circumstances. In such embodiments, the controller 702 activates the requested ultrasonic energy mode using the energy provided by the local generator 810.

After the controller 702 has activated the requested energy mode using the energy provided by the local generator 810 in block 928, the method 900 advances to block 930. In block 930, the controller 702 monitors operation of the local generator 810. That is, similar to the monitoring of the remote generator 106 discussed above, the controller 702 monitors the performance of the requested energy mode provided by the local generator 810. Such monitoring may include, for example, monitoring the amount or level or energy provided by the local generator 810, monitoring the process steps performed by the local generator 810 during the requested energy mode, and/or other operational characteristics of the local generator 810 and/or the energy provided by the local generator 810.

While monitoring the operation of the local generator 810 in block 930, the controller 702 determines whether to supplement the energy mode performed by the local generator 810 with energy provided by the remote generator 106 in block 932. For example, the controller 702 may monitor the sufficiency of the energy provided by the local generator 810 throughout the performance of the requested energy mode and determine to supplement the provided energy should the provided energy be below a reference threshold. If so, the method 900 advances to block 934 in which the controller 702 supplements the energy mode provided by the local generator 810 with energy provided by the remote generator 106. For example, the remote generator 106 may operate in a series fashion with the local generator 810 to provide additional energy for the requested energy mode.

After the controller 702 has supplemented the energy mode with energy from the remote generator 106, the method 900 advances to block 936 in which the controller 702 determines whether the energy mode is completed. If not, the method 900 loops back to block 930 in which the controller 702 continues to monitor the operation of the remote generator 106. If, however, the requested energy mode is completed, the method 900 loops back to block 906 of FIG. 9 in which the controller 702 continues to monitor for a request for activation of an energy mode of the energy-based surgical instrument 102.

Referring back to block 930, while monitoring the operation of local generator 810, the controller 702 also determines whether to perform a supplemental function of the energy-based surgical instrument 102 using energy provided by the remote generator 106 in block 938. For example, as discussed above, the controller 702 may be configured to determine whether to perform the supplemental function of the energy-based surgical instrument 102 based on a sensed characteristic of the patient tissue (e.g., a tissue impedance, temperature, and/or compression). Additionally or alternatively, the controller 702 may be configured to determine whether to perform the supplemental function of the energy-based surgical instrument 102 based on an operation characteristic of the local generator 810 (e.g., the usage level of the output of the local generator 810).

If the controller 702 determines to perform the supplemental function using the energy provided by the remote generator 106 in block 938, the method 900 advances to block 940. In block 940, the controller 702 performs the supplemental function using the energy provided by the remote generator 106. For example, the controller 702 may be configured to utilize the energy provided by the local generator 810 to seal the patient's tissue while the energy provided by the remote generator 106 is used to cut the patient's tissue (or vice versa). Again, in such embodiments, the end effector 120 of the energy-based surgical instrument 102 may include both the harmonic blade 130 and the RF electrode(s) 500, and controller 702 may control operation of the harmonic blade 130 using the energy provided by the remote generator 106 to transect the patient's tissue and control operation of the RF electrode(s) 500 using the energy provided by the local generator 810 to contemporaneously seal the patient's tissue. Additionally or alternatively, the controller 702 may utilize the energy provided by the remote generator 106 to perform various sub-therapeutic functions of the energy-based surgical instrument 120 such as tissue impedance determination.

After the controller 702 has performed the supplemental function using the energy provided by the remote generator 106 in block 940, the method 900 advances to block 936. As discussed above, the controller 702 determines whether the energy mode is completed in block 936. If not, the method 900 loops back to block 930 in which the controller 702 continues to monitor the operation of the local generator 810. If, however, the requested energy mode is completed, the method 900 loops back to block 906 of FIG. 9 in which the controller 702 continues to monitor for a request for activation of an energy mode of the energy-based surgical instrument 102.

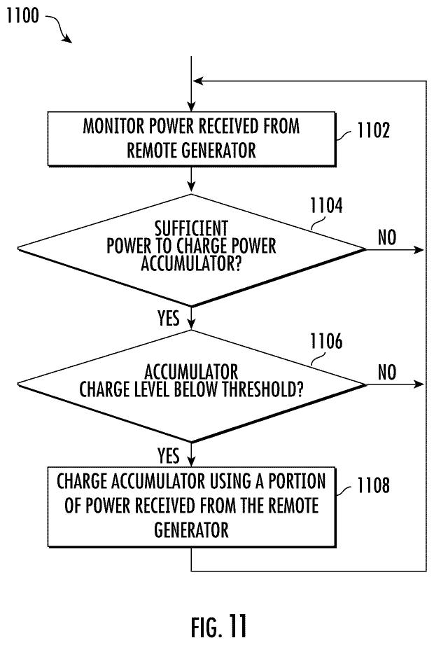

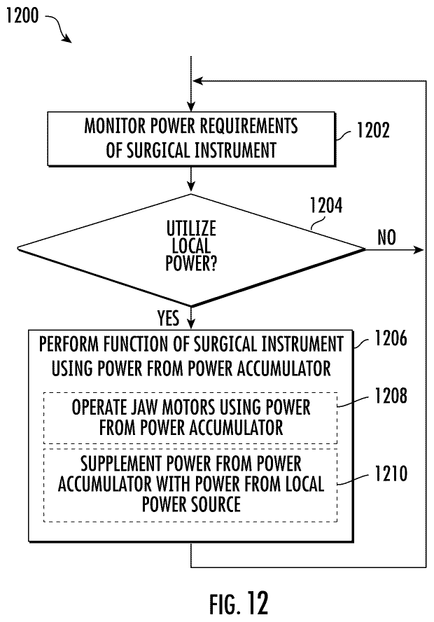

Referring now to FIG. 11, in use, the controller 702 of the energy-based surgical instrument 102 may also execute a method 1100 for charging the power accumulator 830 of the control circuit 800. The method 1100 begins with block 1102 in which the controller 702 monitors power received from the remote generator 106. That is, while the remote generator 106 is providing power to the surgical instrument 102 (e.g., the surgical instrument 102 is not operating solely off of power form the local power source 820), the control circuit 800 monitors the power received from the surgical instrument 102.

In block 1104, the controller 702 determines whether the power received from the remote generator 106 is sufficient enough to charge the power accumulator 830 (e.g., the super capacitor 832). To do so, for example, the controller 702 may compare the present requirements of the functionality of the energy-based surgical instrument 102 (e.g., the power requirements of a requested energy mode) to determine whether there is sufficient, additional power available to charge the power accumulator 830. for example, in some embodiments, the typical functionality of the energy-based surgical instrument 102 may draw an average amount of power (e.g., 7.5 Watts) and, in such embodiments, the controller 702 may determine additional power is available to charge the power accumulator 830 while the present power draw is less than the average amount of power draw.

If the controller 702 determines there is sufficient power to charge the power accumulator 830 in block 1104, method 1100 advances to block 1106. In block 1106, the controller 702 determines whether a present charge level of the power accumulator 830 is below a reference threshold. The reference threshold may be set to any suitable charge level below which the controller 702 is configured to charge the power accumulator 803. For example, the reference threshold may be based on a percentage of total charge of the power accumulator 830. If the controller 702 determines the present charge level of the power accumulator 830 is below the reference threshold, the method 1100 advances to block 1108. In block 1108, the controller 702 charges the power accumulator 830 using a portion of the power received from the remote generator 106. In this way, the controller 702 is configured to charge the power accumulator 830 while ensure there remains enough available power for the functionality of the energy-based surgical instrument 102. After the controller 702 has sufficiently charged the power accumulator 830 using the excess power available from the remote generator 106, the method 1100 loops back to block 1102 in which the controller 702 continues to monitor the power received from the remote generator 106 as discussed above.

Referring now to FIG. 12, in use, the controller 702 may also be configured to execute a method 1200 for performing a function of the energy-based surgical instrument 102 using power from the power accumulator 830. The method 1200 begins with block 1202 in which the controller 702 monitors the power requirements of the energy-based surgical instrument 102. In block 1204, the controller 702 determines whether to utilize local power from the power accumulator 830 based on the present power requirements of the energy-based surgical instrument 102. For example, the controller 702 may monitor for spikes in the power consumption of the energy-based surgical instrument 102 or the request for certain functions that can be performed by presently-available power from the power accumulator 830 such that power from the remote generator 106, the local generator 810, and/or the local power source 820 can be reserved.

If, in block 1204, the controller 702 determines to utilize local power from the power accumulator 830, the method 1200 advances to block 1206. In block 1206, the controller 702 performs one or more functions of the energy-based surgical instrument 102 using power from the power accumulator 830. In some embodiments, only power from the power accumulator 830 may be used to perform the function. For example, in block 1208, the power from the power accumulator 830 may be used to operate the jaw motors 840 that control the jaw assembly 122 of the surgical instrument 102. In other embodiments, the power from the power accumulator 830 may be used to control other motors or perform other function of the surgical instrument 102. Additionally or alternatively, in block 1210, the power from the power accumulator 830 may be used to perform a function of the surgical instrument 102 in conjunction with power from the local power source 820. That is, if the power demands of the particular function of the surgical instrument 102 is greater than either the local power source 820 or the power accumulator 830, the controller 702 may be configured to combine the power from the local power source 820 and the power accumulator 830 to provide a suitable amount of power to perform the functions, while not drawing power form the local generator 810 and/or the remote generator 106. Regardless, after the controller 702 has performed the function of the surgical instrument 102 using power from the power accumulator 830 in block 1206, the method 1200 loops back to block 1202 in which the controller 702 continues to monitor the power requirements of the surgical instrument 102 as discussed above.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A surgical system for performing an energy-based surgical procedure, the system comprising:
an energy-based surgical instrument having an energy mode that, in response to activation, delivers an amount of energy to a patient tissue; and
a remote generator separate from the energy-based surgical instrument and configured to generate a first amount of energy for the energy mode of the energy-based surgical instrument,
wherein the energy-based surgical instrument comprises:
a local generator configured to generate a second amount of energy, less than the first amount of energy of the remote generator, for the energy mode; and
a controller configured to:
monitor for a request to activate the energy mode of the energy-base surgical instrument;
determine whether to use the remote generator or the local generator to activate the energy mode; and
activate the energy mode of the energy-based surgical instrument using the determined generator.

2. The surgical system of claim 1, wherein to determine whether to use the remote generator or the local generator to activate the energy mode comprises to determine whether to use the remote generator or the local generator to activate the energy mode based on the energy-based surgical procedure being performed.

3. The surgical system of claim 1, wherein to determine whether to use the remote generator or the local generator to activate the energy mode comprises to determine whether to use the remote generator or the local generator to activate the energy mode based a sensed characteristic of the patient tissue.

4. The surgical system of claim 1, wherein to determine whether to use the remote generator or the local generator to activate the energy mode comprises to determine to use the remote generator to activate the energy mode,
wherein the controller is further configured to:
monitor the operation of the remote generator;
determine, based on the operation of the remote generator, whether to supplement the first amount of energy generated by the remote generator using the second amount of energy generated by the local generator; and
supplement, in response to a determination to supplement the first amount of energy generated by the remote generator, the first amount of energy generated by the remote generator with the second amount of energy generated by the local generator during the activation of the requested energy mode.

5. The surgical system of claim 1, wherein to determine whether to use the remote generator or the local generator to activate the energy mode comprises to determine to use the remote generator to activate the energy mode,
wherein the controller is further configured to:
monitor the operation of the remote generator;
determine whether to perform a supplemental function of the energy-based surgical instrument using the second amount of energy generated by the local generator; and
perform the supplemental function using the second amount of energy generated by the local generator in response to a determination to perform the supplemental function.

6. The surgical system of claim 5, wherein to determine whether to perform the supplemental function of the energy-based surgical instrument comprises to determine whether to perform the supplemental function of the energy-based instrument based on a sensed characteristic of the patient tissue.

7. The surgical system of claim 5, wherein to determine whether to perform the supplemental function of the energy-based surgical instrument comprises to determine whether to perform the supplemental function of the energy-based instrument based on the operation of the remote generator.

8. The surgical system of claim 1, wherein to determine whether to use the remote generator or the local generator to activate the energy mode comprises to determine to use the local generator to activate the energy mode,
wherein the controller is further configured to:
monitor the operation of the local generator;
determine, based on the operation of the local generator, whether to supplement the second amount of energy generated by the local generator using the first amount of energy generated by the remote generator; and
supplement, in response to a determination to supplement the second amount of energy generated by the local generator, the second amount of energy generated by the local generator with the first amount of energy generated by the remote generator during the activation of the requested energy mode.

9. The surgical system of claim 1, wherein to determine whether to use the remote generator or the local generator to activate the energy mode comprises to determine to use the local generator to activate the energy mode,
wherein the controller is further configured to:
monitor the operation of the local generator;
determine whether to perform a supplemental function of the energy-based surgical instrument using the first amount of energy generated by the remote generator; and
perform the supplemental function using the first amount of energy generated by the remote generator in response to a determination to perform the supplemental function.

10. The surgical system of claim 9, wherein to determine whether to perform the supplemental function of the energy-based surgical instrument comprises to determine whether to perform the supplemental function of the energy-based instrument based on a sensed characteristic of the patient tissue.

11. The surgical system of claim 9, wherein to determine whether to perform the supplemental function of the energy-based surgical instrument comprises to determine whether to perform the supplemental function of the energy-based instrument based on the operation of the local generator.

12. The surgical system of claim 9, wherein the second amount of energy generated by the local generator is used to seal the patient tissue and the first amount of energy generated by the remote generator is used to cut the patient tissue.

13. The surgical system of claim 1, wherein the energy-based surgical instrument further comprises a power accumulator configured to store an amount of power, wherein the controller is configured to charge the power accumulator using a subset of the first energy provided by the remote generator.

14. The surgical system of claim 13, wherein the power accumulator comprises a super capacitor.

15. The surgical system of claim 14, wherein the super capacitor has a capacitance between 0.5 Farads and 2 Farads.

16. The surgical system of claim 13, wherein the controller is configured to determine whether to charge the power accumulator based on an amount of available energy provided by the remote generator.

17. The surgical system of claim 13, wherein the controller is further configured to monitor power requirements of the energy-based surgical instrument and perform a function of the energy-based surgical instrument using power provided by the power accumulator based on the power requirements of the energy-based surgical instrument.

18. The surgical system of claim 17, wherein to perform the function of the energy-based surgical instrument comprises to operate jaw motors of a jaw assembly of the energy-based surgical instrument using the power provided by the power accumulator.

19. The surgical system of claim 17, wherein the energy-based surgical instrument further comprises a local power source and wherein to perform the function of the energy-based surgical instrument comprises to supplement the power provided by the power accumulator with power provided by the local power source.

20. The surgical system of claim 19, wherein to perform the function of the energy-based surgical instrument comprises to operate jaw motors of a jaw assembly of the energy-based surgical instrument using the power provided by the power accumulator and the power provided by the local power source.
